(19) United States
(12) Patent Application Publication
(10) Pub. No.: US 2006/0263326 A1
(43) Pub. Date: Nov. 23, 2006

(54) Freeze-dried scent compound and a method of preparation

(76) Inventor: Mark J. Weiser, Evans City, PA (US)

Correspondence Address: METZ LEWIS, LLC, 11 STANWIX STREET, 18TH FLOOR, PITTSBURGH, PA, 15222, US

(21) Appl. No.: 11/415,462

(22) Filed: May 1, 2006

Related U.S. Application Data
(63) Continuation-in-part of application No. 10/993,654, filed on Nov. 19, 2004.
Application No. 10/993,654 is a division of application No. 09/773,756, filed on Feb. 1, 2001, now abandoned.

(51) Int. Cl. A61K 36/8962; A61K 36/534; A61K 36/55; A61K 36/48; A61K 36/899; A61K 36/47; A61K 36/67; A61K 36/54; A61K 36/53

(52) U.S. Cl. 424/74; 424/725; 424/731; 424/754; 424/750; 424/747; 424/745; 424/757; 424/734; 424/776; 424/739; 424/778; 424/768

(57) ABSTRACT

A freeze-dried scent compound comprises at least one carrier material and at least one active ingredient. The selected carrier material and active ingredient are combined and freeze-dried. The scent compound may be a granular or powdered product. The active ingredient may be at least one of an animal product, a food product, a natural fragrance product or a synthetic fragrance product. The scent compound is freeze-dried which enhances the stability and potency of the scent compound. The scent compound is not significantly affected by environmental conditions and does not significantly evaporate, freeze or absorb into the ground. The scent compound may be used as an attractant, a repellent or an air fragrance material.

### CROSS REFERENCE TO RELATED APPLICATIONS

This application is a continuation in part of co-pending U.S. application Ser. No. 10/993,654, filed Nov. 19, 2004, which is a divisional of abandoned U.S. application Ser. No. 09/773,756, filed Feb. 1, 2001.

### FIELD OF THE INVENTION

The invention relates to a freeze-dried scent compound and its method of preparation. More specifically, this invention relates to the formulation of a scent compound from a carrier material and an active ingredient. The scent compound is freeze-dried to amplify and preserve the odiferous properties of the compound for a significant period of time. The freeze-dried scent compound may be utilized as an attractant, repellant or an air fragrance material.

### BACKGROUND OF THE INVENTION

A scent compound can be used as an attractant, a repellant or an air fragrance product. An attractant is used to attract any type of animal that relies on its sense of smell for food, breeding and defense. Hunters, fishermen and wildlife enthusiasts, including photographers, attempt to attract certain types of animals by using a particular scent that has been duplicated through commercial methods, and this scent must be potent enough to be detected by the animal for a reasonable period of time. Although most animals have a keen and powerful sense of smell that enables them to detect scent at great distances, the scent of a commercial product must be adequately odiferous for a prolonged period of time to be practically effective in attracting the desired animal

A repellent is often utilized to deter a destructive or unwanted animal from entering a designated area. Certain animals use their sense of smell as a primary means of communication. For example, territorial animals communicate their enforced territory through scent marking. Many territorial animals are also predatory. An animal of prey that encounters the scent of a predator understands such a scent to indicate the presence of a predator and equates the marking to the possibility of being devoured as prey. The predator scent creates a fear barrier that an animal of prey attempts to avoid. Therefore, a suitable repellent may emit the scent of a predator of the animal to be repelled from the area where the composition is spread.

Many varieties of attractant and repellent scent mixtures currently exist. For example, Wharton, U.S. Pat. No. 6,682,752, issued Jan. 27, 2004, discloses a Composition for Mole Control that comprises an active ingredient adsorbed onto the solid matrix of a carrier material capable of adsorbing the active ingredient. The active ingredient is castor oil, either as a crude extract or a refined oil from castor seeds. Wharton states that the most suitable adsorbent is a coarse-grained material that is capable of uniform adsorption of the active ingredient. The active ingredient is mixed with the adsorbent and further dried. The composition is spread to infested areas, either by hand or through a mechanical spreader, such as that used to apply fertilizer.

Wharton states that the carrier and active ingredient are mixed together and dried. Natural or air drying may be used to dry a material. However, this process is time-consuming and the active ingredient is not effectively adsorbed into the carrier material. In addition, if the carrier material and active ingredient are dried in the sun, the ultraviolet rays may cause significant damage or degradation to the active ingredient. Another drying method is convection drying, which involves the application of dry heated air to a material. Convection drying causes the evaporation of the surface water, which is replaced by water from inside the solid until the whole material has dried out. One disadvantage of convection drying is that the process generally takes a significant amount of time. Most significantly, convectional drying techniques are often too harsh, often causing significant degradation in the quality of the materials being subjected to the drying process. Further, as a practical matter, convection drying is never complete and leaves some trace water content present in the mixture.

Christenson, II, U.S. Pat. No. 4,944,940, issued on Jul. 31, 1990, discloses a buck lure that comprises a deer tarsal gland and a solution that is predominantly urine. Additional components may include the essence of deer interdigital or femoral glands and any necessary preservatives, such as glycerine, mineral oil and propylene glycol. The lure is intended to mimic the scent of a single deer and cause a dominant buck to protect its territory or alternatively believe a doe has entered his territory. The tarsal gland is most effective when it is fresh. However, the tarsal gland may be dried for long term storage and convenience. The gland may be allowed to dry naturally, or it may be subjected to convectional heating or freeze-drying. The user must rehydrate the tarsal gland with the solution before use.

Similarly, Collara, et al., U.S. Pat. No. 5,896,692 issued Apr. 27, 1999, discloses a freeze-dried scent lure for hunting that is produced from animal urine. The urine can be collected according to gender, time of year or season. Collora discloses that fresh urine is first collected from the species of animal to be hunted or trapped. Next, the urine is freeze-dried to produce a powdered urine product. The freeze-dried urine does not contain any preservatives because preservatives tend to change the natural characteristics of the urine. However, animal glands or glandular secretions may be added to the urine prior to the freeze-drying step. The freeze-dried scent lure may be used in its powdered state or rehydrated with water or liquid urine. Alternatively, the freeze-dried urine may be naturally rehydrated through environmental moisture.

Both Christenson and Collora teach a freeze-dried scent containing only an active scent ingredient. Neither Christenson nor Collora contains any additional product or component that adds potency and/or longevity to the scent emitted by the material or exhibits the durability required of a commercial attractant or a repellent material to provide a long-lasting scent. Moreover, the freeze-dry process leaves microscopic pores in the resulting powder. The pores are created by the ice crystals that sublimate and leave gaps or pores in its place. Liquid solutions which are freeze-dried, such as the Christenson lure, are too easily rehydrated, making them susceptible to environmental factors, such as temperature and moisture. Thus, once the freeze-dried scent lures of Christenson and Collora are placed into the environment or rehydrated, the lures are immediately affected by factors such as temperature and moisture in the environment.

What is lacking in the art, therefore, is an environmentally stable scent compound with enhanced potency and a process to prepare the same that does not result in an alteration of the chemical structure of the scent composition to cause poor product quality and less efficacy.

### BRIEF SUMMARY OF THE INVENTION

A freeze-dried scent compound and its method of preparation are disclosed. The scent compound is in the nature of a carrier material and an active ingredient.

Suitable carrier materials include, but are not limited to, fibrous and other plant materials, naturally occurring minerals that are inherently or physical porous, and a variety of other materials such as dirt, including topsoil, potting soil and the like; shredded paper; natural and synthetic sponge; cloth; wax; corn starch; sodium bicarbonate; magnesium stearate; pumice; activated charcoal; crushed seashells; various types of silica; polymeric materials; psyllium husks; sawdust; salt; and crushed or ground clay. The carrier material can be a combination of two or more carrier materials as well as a single carrier material, as further described herein.

The disclosed freeze-dried scent compound also contains an active ingredient that may be a scent product. The scent product may be an animal product, a food product, a natural fragrance product or synthetic fragrance product. The active ingredient may be a combination of two or more scent products, as well as a single scent product, as further described herein.

The carrier material and the active ingredient of the disclosed scent compound are combined and freeze-dried. The scent compound may be a granular or powdered product. Freeze-drying does not damage the product being dehydrated as with other dehydration methods, which involve higher temperatures. In particular, freeze-drying minimizes, if not eliminates, the molecular damage that the active ingredient may undergo in other dehydration methods. For example, the heat involved in traditional drying techniques may change the shape, texture and the composition of the material being dried. The application of heat energy facilitates chemical reactions in many materials that can significantly change the overall shape, texture and composition of the scent compound.

In contrast, the freeze-drying process does not alter or otherwise destroy the physical or chemical qualities of the scent compound that is being dried. Thus, the carrier material and active ingredient generally remain physically and chemically unchanged. Freeze-drying the scent compound further imparts the characteristic of enhanced stability and potency to the scent compound because the freeze-drying process causes the molecular structure of the active ingredient to be bound to the solid matrix of the selected carrier material. Accordingly, a wide variety of carrier materials can be used in the present invention because the scent compound is not immediately affected by environmental factors, such as moisture and temperature. Moreover, neither the carrier material nor the scent compound will significantly freeze, evaporate or absorb into the ground as may other available scent products.

The freeze-dried scent compound containing the carrier material and active ingredient is less costly than traditional freeze-dried scent materials containing only a scent product. Scent products are generally expensive to obtain because the methods of securing the same are often involved and complicated. Moreover, a significantly large amount of scent product is typically required to produce a small amount of freeze-dried scent material. For example, several gallons of pure animal urine may be required to produce one ounce of freeze-dried animal urine. The carrier material of the scent compound acts as a filler and effectively binds the scent product molecules within solid matrix of the carrier material to provide a greater amount of scent compound by using less scent product. The resulting freeze-dried scent compound exhibits enhanced properties, including greater stability and potency, over the traditional freeze-dried scent materials containing only a scent product.

The scent compound may be stored in a sealed container. If the container containing the scent compound is appropriately sealed, or re-sealed to prevent the reabsorption of moisture, the scent mixture may be stored at room temperature, without refrigeration, and is protected against spoilage almost indefinitely.

The freeze-dried scent compound may be used as an attractant, a repellent or an air fragrance material. In the case of an attractant, the disclosed freeze-dried scent compound may be spread in and around the perimeter of a designated area to encourage an animal to enter the area. Attractants may be used by both hunters and wildlife enthusiasts, including photographers. The scent-compound may also be formulated to mask human odor while hunting or photographing. In the case of a repellent, the scent compound may be spread in and around the perimeter of a designated area to deter animals from entering the area. Repellents may be used in lawn care and gardening to repel animals, such as armadillos, chipmunks, deer, domestic cats, gophers, groundhogs, mice, moles, porcupines, possums, rabbits, raccoons, rats, shrews, skunks, squirrels, voles, woodchucks and insects from areas of growing vegetation or human habitation, including flower beds, golf courses and cemeteries.

The disclosed freeze-dried scent compound may be used as an air fragrance material and may include a natural fragrance product or a synthetic fragrance product as the only active ingredient although natural and synthetic fragrance products may be used in combination with any of the aforementioned scent products and carrier materials as an attractant, a repellent, an air fragrance material or an odor mask.

The disclosed freeze-dried scent compound may be placed directly onto the ground around shrubs or trees, gardens, or flowerbeds. The scent compound may also be placed inside, outside or near a building. The disclosed scent compound may be used in association with a fabric, plastic, polymer or other porous material and positioned in an area to be treated.

The disclosed freeze-dried scent compound provides a natural and environmentally friendly means of attracting certain animals or deterring unwanted animals or insects. The scent compound may contain primarily all natural materials in addition to other materials that do not pose an environmental threat or a threat to humans or animals.

These and other advantages and features of the present invention will be more fully understood upon reference to the preferred embodiments thereof.

### DESCRIPTION OF THE PREFERRED EMBODIMENTS

The freeze-dried scent compound of the present invention contains at least one carrier material and an active ingredient consisting of at least one scent product. The carrier material, as further described herein, refers to a material that acts to adsorb another material. Suitable carrier materials include, but are not limited to, fibrous and other plant materials, naturally occurring minerals that are inherently or physically porous, and a variety of other materials such as dirt, including topsoil, potting soil and the like; shredded paper; natural and synthetic sponge; cloth, such as felt, cotton and the like; wax; corn starch; sodium bicarbonate; magnesium stearate; pumice; activated charcoal; crushed seashells; various types of silica; polymeric materials; psyllium husks; sawdust; salt; and crushed or ground clay.

Suitable examples of fibrous and other plant material that can be used as a carrier material include, for example, straw, hay, seeds, grains, ground corncob, mulch, wood particles and/or compressed wood.

Naturally occurring minerals that are inherently or physically porous may also be used as a carrier material for the formulation. Examples of naturally occurring minerals and rocks may include, for example, calcium carbonate, magnesium carbonate, zeolite, limestone, and small particles of rock. In one embodiment, calcium carbonate is used as a carrier material. Calcium carbonate causes a scent mixture to have longer lasting odor emitting properties when used outdoors because the material is not affected by changes in environmental conditions, such as moisture and temperature. Thus, calcium carbonate may be thought of as a natural time-release carrier material.

In one embodiment, polymeric material may be used as a carrier material. Suitable polymeric materials include, but are not limited to, polystyrenes, polyacrylates, polyacrylamides that are both cross linked and non cross linked, sodium salts of cross linked polyacrylic acid, a co-polymer of cross linked potassium polyacrylate and polyacrylamide, and granular acrylate polymers including a copolymer of polyacrylate and polyacrylamide.

The carrier material can be a combination of two or more carrier materials as well as a single carrier material. Also, the carrier material may be ground before or after it is mixed with the active ingredient, as described herein. The carrier material or scent compound may be ground to a particle size as small as twenty microns but could incorporate larger particle sizes. However, the preferred particle size is smaller than two-thousand microns. Smaller particles sizes may be desirable for aesthetic purposes if the scent compound is used indoors. Larger particles may be desirable if the product is used outdoors. It is to be noted that the stability and potency of the scent compound is not affected by particle size of the carrier material, the active ingredient or the scent compound.

The active ingredient, as further described herein, generally refers to a scent product that is detectable by a human, animal, fish or insect and can be used to attract and/or repel depending on the combination of carrier material and active ingredient. The active ingredient may be a combination of two or more active ingredients as well as a single active ingredient. Suitable active ingredients may include scent products such as a food product or an animal product. A suitable animal product is associated with an animal and emits a distinct odor. Animal products may include, for example, urine, feces, glands and glandular secretions. Other animal bodily secretions, such as bodily fluids collected from a particular animal, may be used as the scent product. Urea, which can either be natural or synthetic, may also be used as the active ingredient scent product.

Most animal products may be thought of as an attractant or a repellent. For example, animal products that are collected from animals such as deer, elk and moose are useful to hunters for attracting an animal that they are trying to hunt. Other animal products, such as those obtained from a wolf, fox, coyote, cougar, lion and tiger are used to repel an animal of prey. In the case of a repellent, the scents associated with the appropriate animal products are understood by animals of prey to be the territorial marking of a predator. Specifically, the animal products associated with a predator animal, such as a coyote, are useful as a repellent to deter animals that are preyed upon by a coyote. Coyote animal products may include urine, glands, glandular secretions, odors, feces and other bodily secretions and are effective at repelling deer, raccoon and elk. Fox animal products are effective in repelling chipmunks, groundhogs, mice, moles, possum, rabbit, rats, shrews, skunks, squirrels, voles and woodchucks.

In one embodiment, the active ingredient in the scent compound is urine. The urine of an animal may contain certain hormones or chemicals that attract other animals of the same species for the purpose of mating or the same may deter an animal from an area. Urine may be collected from the appropriate animal at any time during the year and, in particular, during mating or estrus.

The scent or odor associated with pheromones is used to relay information to another animal. In one embodiment, the pheromone scent associated with a deer gland is the active ingredient in the scent compound. Pheromone scent may be obtained from one or a combination of certain glands or glandular secretions associated glands such as the tarsal gland, the interdigital gland, the forehead gland or the pre-orbital gland.

If the product is to be used indoors or in small confined areas, one or more fragrance scent products may be added as an additional active ingredient to impart a more pleasant odor to the scent compound. However, the fragrance scent product may also be the only active ingredient in the scent compound. A scent compound that contains one or more fragrance scent products as active ingredients may be used as an attractant, a repellent or an air fragrance material.

Suitable fragrance scent products can be natural or synthetic and may include castor oil, cedar oil, cinnamon, cinnamon oil, citric acid, citronella, citronella oil, cloves, clove oil, corn gluten meal, corn oil, cottonseed oil, eugenol, garlic, garlic oil, geraniol, geranium oil, lauryl sulfate, lemongrass oil, linseed oil, malic acid, mint and mint oil, peppermint and peppermint oil, 2-phenethyl propionate, potassium sorbate, putrescent whole egg solids, rosemary, rosemary oil, sesame, sesame oil, sodium chloride, sodium lauryl sulfate, soybean oil, thyme, thyme oil, white pepper, zinc metal strips. The desired fragrance scent product may be combined with other scent products comprising the active ingredient or the fragrance scent product may be added to the scent compound at any time before the scent compound is freeze-dried, as further described herein.

The carrier material and active ingredient of the scent compound may be combined either by hand or by a machine adapted for mixing the carrier material and active ingredient. Under certain circumstances, the carrier material and active ingredient may also be combined together naturally. In one embodiment, fibrous plant material is the selected carrier material and an animal product is the active ingredient. The carrier material and animal product may be naturally combined by using the fibrous plant material as bedding for a particular animal. The resulting combination of carrier material and animal product may be better in quality than a carrier material and animal product combined by hand or machine because the impregnation of the animal product and associated scent onto the carrier material is natural. It should be noted that any of the above-mentioned carrier materials, including calcium carbonate and limestone, may be utilized as bedding material for an animal for the purpose of natural combination of carrier material and active ingredient.

In one embodiment, the scent compound contains at least one animal product as the active ingredient. The animal may urinate, rub, walk, sleep, breed, or otherwise contact the material. Animal products associated with the animal, such as urine, saliva, odors, feces and glandular secretions are collected through the natural use of the bedding material by the animal. Specifically, if a deer uses the selected carrier material or materials as bedding, the animal product or products may include interdigital glandular material from the hoof, urine, tarsal material, feces, body odor, saliva, and other odors or bodily secretions depending on the time of year that the deer is using the bedding material.

Depending on the end use of the freeze-dried scent compound, the ratio of carrier material to active ingredient may be varied to provide scent compounds having different odor emitting properties. The strength of scent, texture of the product, appearance of the product, weight and life of the product in the field can be varied. The carrier material and active ingredient may be combined in a ratio of 70% to 95% carrier material and 5-30% active ingredient, with all proportions based on weight. In the preferred embodiment, the ratio is 95% carrier material to 5% active ingredient. Additional examples of variations of carrier material to active ingredient are described in Weiser, U.S. Pat. No. 6,149,901 issued Nov. 21, 2000.

Certain combinations of carrier materials provide a compound with improved scent properties. Certain scent compounds may be adverse to wet weather or may work better in different humidities or elevations. The carrier material combination of calcium carbonate and limestone is resistant to most environmental conditions. However, a sawdust and potting soil carrier material combination is greatly affected by the environment and are dissipated quickly by precipitation. In one embodiment, a suitable ratio of a carrier material combination containing calcium carbonate and limestone is 75% calcium carbonate and 25% limestone. The carrier combination may then be mixed in a ratio of 70% to 95% carrier material and 5-30% active ingredient. In another embodiment, a suitable ratio of scent product contains 40% calcium carbonate, 40% vermiculite and 20% active ingredient with all proportions based on weight. In yet another embodiment, a possible combination is 30% calcium carbonate, 20% vermiculite, 20% topsoil, 20% sawdust and 10% active ingredient with all proportions based on weight.

The scent compound is subjected to a freeze-drying process to form a granular or powdered product. Freeze-drying, or lyophilization, is a dehydration process used to preserve a material. The freeze-drying process causes the molecular structure of the active ingredient to be bound to the solid matrix of the carrier material. Thus, Any of a wide variety of carrier materials can be used in the scent compound. The scented compound can be frozen remotely using either dry ice, occurring at a temperature of approximately −78° C., or liquid nitrogen, occurring at a temperature of approximately −196° C., to provide the necessary physical condition or the scented compound may be freeze-dried using a freeze-drying apparatus, which has the ability to freeze a substrate through refrigeration. Freeze-drying works by freezing the material and then reducing the surrounding pressure to allow the frozen water in the material to sublimate directly from the solid phase to the gas phase. A typical freeze-drying machine, such as the Lyolab 984 Micro-Controller, manufactured by American Lyophilizer, Inc., consists of a freeze-drying chamber with several shelves attached to heating units, a freezing coil connected to a refrigerator compressor, and a vacuum pump.

The freeze-dry process of the scent compound includes the following fundamental steps. First, the scent compound is frozen to provide a necessary physical condition for low temperature drying. The unfrozen scent compound is placed onto at least one of the several shelves of the freeze-drying chamber. The chamber is sealed and the refrigerator compressor is initialized which causes the lowering of the temperature inside the freeze-drying chamber. The scent compound becomes frozen solid and the water contained within the scent compound, if any, is separated from the carrier material and active ingredient on a molecular level. The water becomes formed ice within the scent compound.

Next the scent compound is placed under vacuum to enable the frozen solvent in the scent compound to vaporize without passing through the liquid phase, a process known as sublimation. The sublimation process should be controlled to ensure that the appropriate amount of water vapor is produced. The pump system of the freeze-dry chamber may be unable to remove any excess water vapor, which may cause the material to be rehydrated and degraded in quality. The vacuum pump is initialized and air is forced out of the freeze-drying chamber by lowering the atmospheric pressure.

During this period of low atmospheric pressure, the heating units apply a small amount of heat to the shelves which causes the formed ice to change phase. The heating units must be adequately controlled to provide the proper amount of heat because overheating the scent compound may cause a change in the composition or structure of the same. As the heat is applied, the formed ice changes phase directly into a water vapor because the pressure inside the freeze-drying chamber is so low. As the water vapor flows from the freeze-drying chamber, it condenses onto the freezing coil in a solid ice form.

Once the scent compound is properly dried, it is sealed in a moisture free container. An oxygen or moisture absorbing material, such as a dessicant, may also be added to the container. A suitable absorbing material may be silica gel. As long as the container is permanently sealed or properly resealed after use, the scent compound can be stored almost indefinitely without a degradation in quality of material or scent potency.

The freeze-dried scent compound is a powder material that may be used as an attractant, a repellent or an air fragrance material. In one embodiment, the scent compound is placed directly onto the ground by lightly sprinkling the same around the perimeter of the designated area. The area may be a group of shrubs or trees, a garden or a flowerbed. In another embodiment, the scent compound may be placed near small cracks or openings where an animal may enter including, but not limited to, along the foundation, inside or under a house, garage, shed or any other building. The small cracks or entrance to the opening may be sealed once the animal leaves the area. In yet another embodiment, if the scent compound is utilized to repel a tunneling animal, the scent compound may be placed directly into the tunnel created by the animal. The entrance to the tunnel may be covered with dirt, or the like, to discourage the animal from re-entering the tunnel.

The scent compound may also be used in association with a fabric, plastic, polymer or other porous material through which a scent may be emitted. In one embodiment, the compound is placed inside a nylon mesh material. The ends of the nylon mesh are closed and the container is placed in an area to be treated. In another embodiment, a hunter or wildlife enthusiast may attach the nylon mesh containing the scent-compound to his shoes or body to mask human odor.

Although the scent compound should emit a detectable scent for a prolonged period of time, the scent compound may need to be renewed, as needed.

While the present preferred embodiment of the invention is described, it is to be distinctly understood that the invention is not limited thereto, but may be otherwise embodied and practiced with the scope of the following claims.

What is claimed:

1. A scent compound comprising:
one or more carrier materials and one or more active ingredients selected from the group consisting of food products, animal products, natural fragrance products and synthetic fragrance products,
said carrier material and active ingredient being combined and freeze-dried.

2. A scent compound according to claim 1, wherein said one or more carrier materials being selected from the group consisting of fibrous and other plant material, naturally occurring minerals, and a variety of other materials such as dirt, including topsoil, potting soil and the like; shredded paper; natural and synthetic sponge; cloth; wax; corn starch; sodium bicarbonate; magnesium stearate; pumice; activated charcoal; crushed seashell; various types of silica; polymeric material; psyllium husk; sawdust; salt; and crushed or ground clay.

3. A scent compound according to claim 2, wherein said fibrous and other plant material being selected from the group consisting of straw, hay, seeds, grains, ground corncob, mulch, wood particles and compressed wood.

4. A scent compound according to claim 2, wherein said naturally occurring minerals being selected from the group consisting of calcium carbonate, magnesium carbonate, zeolite, limestone, and pumice.

5. A scent compound according to claim 1, wherein said one or more carrier materials comprises calcium carbonate.

6. A scent compound according to claim 1, wherein said one or more carrier materials comprises limestone.

7. A scent compound according to claim 2, wherein said polymeric material being selected from the group consisting of polystyrenes, polyacrylates, polyacrylamides, sodium salts of a cross-linked polyacrylic acid, co-polymers of cross-linked potassium polyacrylate and polyacrylamide and granular acrylate polymers.

8. A scent compound according to claim 1, wherein said animal products being selected from the group consisting of urine, feces, glands, glandular secretions, unknown bodily fluids and urea.

9. A scent compound according to claim 1, wherein said one or more active ingredient comprises synthetic urea.

10. A scent compound according to claim 1, wherein said natural fragrance product being selected from the group consisting of: castor oil, cedar oil, cinnamon, cinnamon oil, citric acid, citronella, citronella oil, cloves, clove oil, corn gluten meal, corn oil, cottonseed oil, eugenol, garlic, garlic oil, geraniol, geranium oil, lauryl sulfate, lemongrass oil, linseed oil, malic acid, mint and mint oil, peppermint and peppermint oil, 2-phenethyl propionate, potassium sorbate, putrescent whole egg solids, rosemary, rosemary oil, sesame, sesame oil, sodium chloride, sodium lauryl sulfate, soybean oil, thyme, thyme oil, white pepper and zinc metal strips.

11. A scent compound according to claim 1, wherein said synthetic fragrance product being selected from the group consisting of: castor oil, cedar oil, cinnamon, cinnamon oil, citric acid, citronella, citronella oil, cloves, clove oil, corn gluten meal, corn oil, cottonseed oil, eugenol, garlic, garlic oil, geraniol, geranium oil, lauryl sulfate, lemongrass oil, linseed oil, malic acid, mint and mint oil, peppermint and peppermint oil, 2-phenethyl propionate, potassium sorbate, putrescent whole egg solids, rosemary, rosemary oil, sesame, sesame oil, sodium chloride, sodium lauryl sulfate, soybean oil, thyme, thyme oil, white pepper and zinc metal strips.

12. A scent compound according to claim 1, wherein said scent compound comprises approximately 95% carrier material by weight and 5% active ingredient by weight.

13. A method of preparing a scent compound comprising:
obtaining a combination of: (i) one or more carrier materials and (ii) one or more active ingredients selected from the group consisting of food products, animal products, natural fragrance products and synthetic fragrance products,
combining said one or more carrier materials and said one or more active ingredients, and
freeze-drying the combination of said one or more carrier materials and said one or more active ingredients.

14. A method of preparing a scent compound according to claim 13, wherein said one or more carrier materials being selected from the group consisting of: fibrous and other plant materials, naturally occurring minerals, and a variety of other materials such as dirt, including topsoil, potting soil and the like; shredded paper; natural and synthetic sponge; cloth; wax; corn starch; sodium bicarbonate; magnesium stearate; pumice; activated charcoal; crushed seashells; various types of silica; polymeric materials; psyllium husks; sawdust; salt; and crushed or ground clay.

15. A method of preparing a scent compound according to claim 13, wherein said one or more carrier materials comprises calcium carbonate.

16. A method of preparing a scent compound according to claim 13, wherein said one or more carrier materials comprises limestone.

17. A method of preparing a scent compound according to claim 13, wherein said animal products being selected from the group consisting of urine, feces, glands, glandular secretions, unknown bodily fluids and urea.

18. A method of preparing a scent compound according to claim 13, wherein said one or more active ingredient comprises synthetic urea.

19. A method of preparing a scent compound according to claim 13, wherein said natural fragrance product being selected from the group consisting of: castor oil, cedar oil, cinnamon, cinnamon oil, citric acid, citronella, citronella oil, cloves, clove oil, corn gluten meal, corn oil, cottonseed oil, eugenol, garlic, garlic oil, geraniol, geranium oil, lauryl sulfate, lemongrass oil, linseed oil, malic acid, mint and mint oil, peppermint and peppermint oil, 2-phenethyl propionate, potassium sorbate, putrescent whole egg solids, rosemary, rosemary oil, sesame, sesame oil, sodium chloride, sodium lauryl sulfate, soybean oil, thyme, thyme oil, white pepper and zinc metal strips.

20. A method of preparing a scent compound according to claim 13, wherein said synthetic fragrance product being selected from the group consisting of: castor oil, cedar oil, cinnamon, cinnamon oil, citric acid, citronella, citronella oil, cloves, clove oil, corn gluten meal, corn oil, cottonseed oil, eugenol, garlic, garlic oil, geraniol, geranium oil, lauryl sulfate, lemongrass oil, linseed oil, malic acid, mint and mint oil, peppermint and peppermint oil, 2-phenethyl propionate, potassium sorbate, putrescent whole egg solids, rosemary, rosemary oil, sesame, sesame oil, sodium chloride, sodium lauryl sulfate, soybean oil, thyme, thyme oil, white pepper and zinc metal strips.
